(19) United States
(12) Patent Application Publication
(10) Pub. No.: US 2005/0238632 A1
(43) Pub. Date: Oct. 27, 2005

(54) Propellant formulations

(76) Inventors: David S. Alburty, Drexel, MO (US); Kelly L. Brown, Kansas City, MO (US); Andrew E. Page, Kansas City, MO (US); Michael F. Fischer, Lee's Summit, MO (US)

Correspondence Address: BLACKWELL SANDERS PEPER MARTIN LLP, 720 OLIVE STREET, SUITE 2400, ST. LOUIS, MO, 63101, US

(21) Appl. No.: 10/830,514

(22) Filed: Apr. 23, 2004

(51) Int. Cl. A01N 63/00; A61L 9/04

(52) U.S. Cl. 424/93.46

(57) ABSTRACT

The present invention provides propellant formulations for non-pharmaceutical use in dispersing insoluble particles having biological activity, such as bacterial spores and/or biological analogues, using a dispersion device such as a metered-dose inhaler. It is preferred that the propellant formulations of the present invention are chemically compatible with the biological analogues with which they are to be used, have substantially the same specific gravity as the biological analogues, and have sufficient vapor pressure to prevent agglomeration of the biological analogues. Methods of dispersing said biological analogues in accordance with the present invention are also provided.

### BACKGROUND OF INVENTION

The present invention relates generally to an aerosol propellant formulation, and more particularly to an aerosol propellant formulation for use with insoluble particles having biological activity, such as bacterial spores and biological analogues.

Aerosol propellant formulations are well known in the art. Such formulations have been used to administer drugs for decades. For such purposes, the formulations are generally prepared by dispersal of the drug within the selected propellant. The propellant formulation containing the drugs is then filled into canisters suitable for delivering pharmaceutical aerosol formulations. Canisters generally include a container capable of withstanding the vapor pressure of the propellant used, such as, for example, a plastic, plastic-coated, or metal canister. The canister is preferably coupled with a metering valve. The metering valves are designed to deliver a metered amount of the formulation per actuation. Such metered dose inhalers (MDIs) are well known in the art.

In recent years, as the level of sophistication in the field of biotechnology has increased, the threat of biological weapon use by terrorist groups or rogue nations has also increased. Thus, it has become imperative that means for the early detection and identification of biological warfare agents are in place. It is undesirable, however, to release potential biological warfare agents, such as anthrax, into a facility or an outdoor environment for the purpose of testing or calibrating equipment designed to detect biological warfare agents. Further, use of non-lethal surrogate organisms, such as Bacillus globigii, which can be used as a surrogate for anthrax, still presents risks of infection, allergic response, contamination and the like. It is most desirable, therefore, to use non-living biological analogues for the testing and calibration of biological warfare agent detection systems.

In order to use biological analogues for the purposes described above, there must be in place some means of disseminating or dispersing the biological analogue into the environment in controlled amounts. This should be done in such a way as to simulate dispersion of an actual biological warfare agent in the event of an attack or contamination so that the efficacy of the detection system can be assessed accordingly. One means of dispersing biological analogues is through the use of a metered dose inhaler mechanism.

Providing a propellant formulation suitable for use with biological analogues presents a number of challenges. The formulation must be chemically compatible with the microscopic “bead” portion of the biological analogue, which may be constructed from polystyrene and the like. The formulation must also be biochemically compatible with the active portions of the biological analogue. For example, the formulation may need to be compatible with DNA molecules, or with streptavidin-biotin bonds present on the biological analogue. In addition to compatibility with the biological analogue, the formulation must be compatible with the valve and canister of the metered dose inhaler used.

Further, the formulation must provide neutral buoyancy for the biological analogue so that the biological analogues are evenly suspended in the formulation. The vapor pressure of the formulation must also be such that it provides for dissemination of the biological analogues as individual particles rather than agglomerations, yet retains compatibility with the metered dose inhaler. It is also desirable that the propellant formulation be nontoxic and nonflammable.

A propellant formulation that meets the above requirements is greatly needed.

### SUMMARY OF INVENTION

The present invention provides propellant formulations for use in the dispersion for non-pharmaceutical uses of insoluble particles having biological activity, such as bacterial spores and/or biological analogues, via a metered dose inhaler or other dispersion mechanism.

In a preferred embodiment of the present invention, the formulations of the present invention are biochemically compatible with the insoluble particles and have a specific gravity substantially the same as that of the insoluble particles. The insoluble particles may be bacterial spores, such as, for example, Bacillus globigii spores, or may be biological analogue or other particles.

A preferred embodiment of the present invention is biochemically compatible with biological analogues comprising at least one DNA molecule attached to a polystyrene microsphere via a streptavidin-biotin interaction. This preferred embodiment of the present invention is also chemically inert with respect to polystyrene and the other components of the biological analogue and MDI.

It is preferred that the propellant formulations of the present invention have substantially the same specific gravity as the biological analogues with which they are used so that the biological analogues remain evenly suspended in the propellant for a prolonged period after mixing. For example, with respect to the biological analogues comprising a DNA molecule attached to a polystyrene bead via a streptavidin-biotin bond, it is preferred that the specific gravity of the propellant formulation is about 1.06.

It is also preferred that the propellant formulations of the present invention have a vapor pressure that provides good dispersion of biological analogues from the MDI valve. For example, with respect to a biological analogue comprising a DNA molecule attached to a polystyrene bead via a streptavidin-biotin bond as described above, it is preferred that the propellant formulation have a vapor pressure of over approximately 50 psia at 70° Fahrenheit.

One embodiment of the present invention includes, by weight, about 85.40% 1,1,1,2-tetrafluroethane (HFA-134A), about 10.5% isobutane, about 3.46% ethanol, and about 0.63% propylene glycol (1,2-propanediol).

Another embodiment of the present invention includes a pre-mixed propellant blend which may be added to a metered-dose inhaler already containing biological analogues in a carrier such as propylene glycol. One embodiment of the pre-mixed propellant blend includes, by weight, about 85.94% tetrafluoroethane, about 10.54% isobutane, and about 3.48% ethanol.

Another embodiment of the present invention provides a method for dispersing a plurality of biological analogues. The method includes providing the biological analogues in a carrier, placing the biological analogue/carrier suspension in a metered-dose inhaler, providing a propellant formulation having a specific gravity substantially the same as that of the plurality of biological analogues, adding the propellant formulation to the metered-dose inhaler so that the biological analogues disseminate therein, and actuating the metered-dose inhaler in order to disperse the biological analogues.

Another embodiment of the present invention includes a mixture of isobutane and HFA 134a having a specific gravity of about 1.006 for use in dispersing dry B. globigii spores.

It is preferred, with respect to the above method, that the propellant formulation have a vapor pressure sufficient to prevent agglomeration of the plurality of biological analogues, and that the propellant formulation is chemically compatible with the biological analogues.

### DETAILED DESCRIPTION

The present invention provides novel propellant formulations for use in the dispersion of insoluble particles having biological activity, such as bacterial spores and/or biological analogues. The particles dispersed are for non-pharmaceutical uses.

As used herein, the term “non-pharmaceutical” means uses other than those that provide medicaments to a person for the purpose of treating diseases or other medical conditions. The present formulation may be used with a metered-dose inhaler or other dispersion system. For any given biological analogue or other particle, the formulations of the present invention must have the proper physical characteristics, such as specific gravity, vapor pressure, and chemical compatibility.

The term “biological analogue,” as used herein, refers to any artificial particle, device, or composition that functions to simulate a biological organism or some aspect of a biological organism. Such a biological analogue may include, but is not limited to, polystyrene or glass beads having DNA, proteins, or other biomolecules attached thereto for the purpose of simulating an actual biological organism.

The term “specific gravity,” as used herein, is defined such that the specific gravity of a substance is the ratio of the density of that substance to the density of water at the same temperature.

The term “chemical compatibility,” as used herein, refers to compatibility between a biological analogue, including its component parts, chemical bonds and the like, and the present propellant formulations and their individual components, such that, when used in accordance with the teachings of the present invention, the propellant formulations do not destroy or alter the chemical structure or composition of the biological analogues, or in any other way render the biological analogues unsuitable for their intended purpose.

The discussion below is directed primarily to biological analogues, but the principles set forth apply to other particles are described herein.

When biological analogues are dispersed, such as, for example, by a metered-dose inhaler, it is desirable that the quantity of biological analogues dispersed remain constant between dispersion events. To that end, it is desirable that a propellant formulation used for the dispersion of biological analogues from a metered-dose inhaler have substantially the same specific gravity as the biological analogue itself. This allows the biological analogues to remain evenly suspended in the propellant formulation for extended periods after mixing. Since it is unlikely that any given propellant will have the same specific gravity as a biological analogue, it is preferred that a propellant formulation for dispersing biological analogues contain at least two propellants—one having a specific gravity greater than that of the biological analogue, and another having a specific gravity less than that of the biological analogue. The combination of at least two propellants, in the appropriate proportions, results in a propellant formulation having the desired specific gravity. In certain cases, a third solvent may be needed to cosolve the liquid in which the biological analogues are originally prepared.

In theory, the propellants used in the present formulations may be selected from a broad range of propellants. For example, any fluorocarbon may, in theory, be useful as a propellant for dispersing biological analogues. For any given biological analogue, however, it is important that the chemical compatibility between the propellants and the biological analogue be maintained. With that in mind, suitable propellants may include tetrafluoroethane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and the like, or mixtures thereof. Any of these, or any combination thereof, may provide the appropriate chemical compatibility needed with respect to any given biological analogue. This list of propellants is, however, exemplary and is not limiting with respect to the present invention.

It is also desirable that the propellant formulations of the present invention have sufficient vapor pressure to disperse biological analogues as individual particles rather than agglomerations of particles. If the biological analogues are dispersed as agglomerations, the resulting aerodynamic diameter of the particles is affected. The change in aerodynamic diameter may render testing or calibration of biological weapon detections systems unreliable because the performance of the system is not being assessed using particles that truly mimic the desired biological organism. This can occur because the resulting agglomeration is not ‘detected’ by the detection system as the appropriate organism, or because the biological analogue agglomeration is not disseminated in a way that mimics the actual organism in question, or for various other reasons or combinations of reasons. The vapor pressure of the propellant of the present invention must, however, be within the tolerances of the metered-dose inhaler, or other dispersion device, in order to be used in the dispersion of the biological analogues. If the vapor pressure is too great, the metered-dose inhaler or other dispersion device may be damaged.

A preferred embodiment of the present invention is formulated for use with biological analogues having a polystyrene bead and further having genomic DNA attached thereto via streptavidin-biotin linkages. Specifically, this preferred embodiment of the present invention is formulated for use with such biological analogues having Bacillus globigii DNA attached thereto. These particular biological analogues are referred to below as BioSim® Bg biological analogues (Sceptor Industries, Inc., Kansas City, Mo.), or simply as Bg biological analogues. Unless otherwise indicated, BioSim® Bg biological analogues were used for the Bg biological analogues in the experiments below. The DNA used for such biological analogues could, however, be obtained from any source, and may include synthetic and/or naturally occurring DNA.

The preferred embodiment of the present invention formulated for use with Bg biological analogues includes, by weight, about 85.40% tetrafluoroethane, about 10.51% isobutane, about 3.46% ethanol, and about 0.63% propylene glycol.

In order to determine the usefulness of this formulation with respect to the Bg biological analogues described above, a number of tests were conducted. Though the tests were conducted specifically with respect to a preferred formulation for use with Bg biological analogues, the principles derived from these experiments may be applicable to other embodiments of the present invention. The following examples are not intended to limit the scope of the present invention.

### EXAMPLE 1

The Bg biological analogues used were provided in propylene glycol as a carrier (which accounts for the percent, by weight, propylene glycol included in the formulation as set forth above). Because of the use of propylene glycol as a carrier, the miscibility of the propellant formulation, particularly with respect to the miscibility of propylene glycol with tetrafluoroethane and isobutane, had to be determined. In addition, since the Bg biological analogues used included a polystyrene bead, the chemical compatibility of the propellant formulation and polystyrene had to be determined.

Clear pressure jars were filled with the propellant formulation. Ethanol was used as a cosolvent to cosolve the propylene glycol in the formulation. The results showed that only a small amount of ethanol, as set forth in the formulation described above, was needed to accomplish this. Thus, with the use of a cosolvent, the miscibility of the propylene glycol carrier in the tetrafluoroethane and ethanol formulation was sufficient for purposes of the present invention. Although ethanol was used as a co-solvent in this example, it is contemplated that other light alcohols, such as any one-, two-, three-, or four-carbon alcohols may be used as appropriate with any given formulation/particle combination.

It is known that polystyrene beads can dissolve or change size and/or shape due to chemical incompatibility. In order to determine the chemical compatibility of the propellant formulation and polystyrene, 10 mm polystyrene pellets were placed in the formulation blend and stored in a glass observation jar. These pellets did not visibly change over the course of an twelve-month observation period. Thus, the 10 mm polystyrene pellets demonstrated sufficient chemical compatibility with this embodiment of the present invention to be suitable for use with the present invention.

### EXAMPLE 2

To test the effects of the propellant formulation and dispersing mechanism on smaller polystyrene beads, 5.5 μm polystyrene beads were suspended in MDIs containing the propellant formulation. These beads were made of the same polystyrene material as the Bg biological analogues described above, but without the streptavidin coating or DNA attachment. Thus, the effects of the propellant formulation on the polystyrene alone could be observed. The beads were disseminated into a Biological Detection System (BDS) test rig. The beads were then analyzed using a Coulter Multisizer™ II particle sizer/counter (Beckman Coulter, Inc., Fullerton Calif.). The test rig included an BDS inlet hood, flexible tubing, BDS precyclone and SpinCon® (Sceptor Industries, Inc., Kansas City, Mo.). The tests showed that the beads were not harmed by either the propellant or dispensing system and could be collected by the BDS.

### EXAMPLE 3

In order to test the effects of the propellant formulation and dispersing mechanism on an actual biological analogue, 0.95 μm Bg biological analogues were suspended in MDIs containing a preferred embodiment of the present propellant formulation, as described above. Samples were then analyzed using a one-bay GeneXpert® (Cepheid, Sunnyvale, Calif.). These samples tested positive for Bg. Thus, neither the propellant formulation nor the dispersing mechanism harmed the Bg biological analogues. The Bg biological analogues were able to be collected by the BDS and analyzed by GeneXpert®. A twelve-week stability study was conducted, showing consistent operation and function of the Bg biological analogue MDIs over that time period. Subsequent tests showed that the MDIs can be consistently prepared using the described propellant blend.

It is generally preferred that, when using a metered-dose inhaler as a dispersion device, the biological analogues be placed within the metered-dose inhaler while suspended in a carrier, such as, for example, propylene glycol, prior to the addition of the propellant formulation to the metered-dose inhaler. It is then preferred that the other components of the propellant formulation, including a co-solvent if necessary, be added to the metered-dose inhaler after the addition of the biological analogues and their carrier. In the embodiment of the present invention directed to dispersion of Bg biological analogues, for example, the Bg biological analogues are suspended in propylene glycol and the suspension is placed within an empty metered-dose inhaler. A propellant blend including, by weight, about 85.94% tetrafluroethane, about 10.58% isobutane, and about 3.48% ethanol as a co-solvent, is then added to the metered-dose inhaler. Pre-mixing the propellant formulation in this manner reduces possible degradation of the biological analogues that can occur if concentrated alcohol is added directly to the biological analogues.

### EXAMPLE 4

### Stability Study

To further demonstrate the utility of the present invention with respect to dispensing biological analogues and the like, the following tests were conducted:

A metered dose of biological analogues was dispensed using an MDI and a propellant formulation produced in accordance with the teachings of the present invention. The biological analogues were dispensed in one end of an intake plenum. The intake plenum was sealed to a counter top with duct tape, and a metal block was used to align the MDI with the cross-sectional center of the intake plenum. The plenum was connected to a BDS cyclone preseparator by a five foot long, 1.25 inch internal diameter anti-static hose running from a flange at one end of the plenum to the preseparator. A seven foot length of the same type of hose was used to connect the cyclone to a SpinCon,® which was set to draw in 400 lpm of air at the inlet with water in the contactor, and to provide a sample volume of about 10 ml. The fluidics module was also placed in the hood and the sample reservoir was bypassed with the extraction pump connected directly to the 15 ml centrifuge tubes used for sample recovery.

Prior to running each individual test, the system was decontaminated with 10% bleach and thoroughly rinsed with distilled water to prevent possible bleach contamination. The sample vials were tare weighed, 1 ml of polyethylene glycol added, and the vials were re-weighed and connected to the fluidics module. Once started, the system indicated “sampling,” and the SpinCon® sampled ambient air through the plenum for one minute. The MDI was then discharged into the system. The SpinCon® ran for an additional minute and was then shut down and the sample recovered. To test background conditions, the SpinCon® sampled ambient air for two minutes without an MDI charge.

All samples were analyzed on a Cepheid GeneXpert® PCR machine using Ba 4-plex cartridges. The back right well in each sample was filled with 350 ml of molecular-grade water in place of the standard 0.1% bleach buffer, and the front center well in each sample was filled with 3.5 ml of Buffer 2. The sample acquired from the SpinCon® was then vortexed for about ten seconds and 1 ml was placed in the sample well. For analysis, the Ba 4-plex cartridge was treated as a Bg-duplex cartridge by the GeneXpert.®

The results of the above-described experiments were as follows:

| | | | Net | | |
|---|---|---|---|---|---|
| Cannis- | Test | Number of | Weight | GeneXpert ® | Concentration |
| ter | Number | Sprays | (g) | Result | Threshold |
| Blank | | — | 10.23 | NEG | 0 |
| | | — | 9.08 | NEG | 0 |
| 0 | — | 5 | 10.07 | POS | 41.82 |
| | | | 10.07 | POS | 42.47 |
| 1 | 1 | 5 | 9.56 | POS | 34.70 |
| | 2 | 5 | 9.63 | POS | 36.26 |
| | 3 | 5 | 9.82 | POS | 33.89 |
| | 4 | 7 | 9.10 | POS | 33.37 |
| 2 | 1 | 5 | 9.42 | POS | 34.72 |
| | 2 | 5 | 11.03 | POS | 33.15 |
| | 3 | 7 | 10.65 | POS | 34.14 |
| 3 | 1 | 5 | 9.31 | POS | 33.63 |
| | 2 | 5 | 10.64 | POS | 33.72 |
| 5 | 1 | 5 | 10.46 | POS | 35.06 |
| | 2 | 5 | 8.86 | NEG | 0.00 |
| | 3 | 5 | 10.58 | POS | 33.02 |
| | 4 | 5 | 10.58 | POS | 34.40 |
| 7 | 1 | 5 | 10.41 | POS | 34.36 |
| | 2 | 5 | 9.58 | POS | 35.86 |
| | 3 | 5 | 10.57 | POS | 35.16 |
| 8 | 1 | 5 | 10.25 | POS | 37.88 |
| | 2 | 5 | 9.58 | POS | 32.60 |
| | 3 | 5 | 10.92 | POS | 37.55 |
| 9 | 1 | 5 | 9.57 | POS | 36.47 |
| | 2 | 5 | 7.65 | POS | 35.90 |
| | 3 | 5 | 8.49 | POS | 34.31 |
| 10 | 1 | 5 | 8.20 | NEG | 0.00 |
| | 2 | 5 | 7.28 | POS | 34.25 |
| | 3 | 5 | 9.97 | POS | 33.75 |

This data indicates that the Bg biological analogues used work with the formulation described above.

### EXAMPLE 5

### Dry Bg Dissemination and Stability Study

For this study, MDIs were filled with 100 mg of dry Bg powder and approximately 7 mL of a mixture of isobutane and HFA 134a having a specific gravity of about 1.006 g/mL. Three puffs (each puff being one actuation of the MDI valve) from each MDI were released into a flow tube and collected in an individual AGI-30 (all-glass impinger). The collected samples were then plated, colonies counted, and colony-forming units (CFU) per puff calculated

The results of this study were as follows:

| | | | | | | AGI-30 | |
|---|---|---|---|---|---|---|---|
| | Plate | Plate | Plate | | Dilution | Volume | |
| Run | 1 | 2 | 3 | Average | (1:x) | (mL) | CFU/puff |
| Initial P1 | 205 | 227 | 192 | 208 | 1000 | 19 | 1.3E + 07 |
| Later P1 | 226 | 243 | 239 | 236 | 1000 | 19 | 1.5E + 07 |
| Later P2 | 217 | 237 | 220 | 225 | 1000 | 19 | 1.4E + 07 |
| Later P3 | 191 | 218 | 176 | 195 | 1000 | 19 | 1.2E + 07 |
| New P1 | 180 | 147 | 227 | 185 | 1000 | 19 | 1.2E + 07 |
| New P2 | 199 | 192 | 189 | 193 | 1000 | 19 | 1.2E + 07 |
| New P3 | 153 | 141 | 164 | 153 | 1000 | 19 | 9.7E + 06 |

P1, P2 and P3 refer to puff 1, puff 2 and puff 3, respectively. As the above data indicates, the Bg particles present in the dry powder were successfully and stably disseminated and collected.

The following table provides data from an MDI dilution series. The dilution series compares well with theoretical values, though there was likely some loss in the flow tube and collection system.

| | | AGI | | | | |
|---|---|---|---|---|---|---|
| | AGI | Volume | | Time | Rate | |
| Puffer | Number | (mL) | CFU/mL | (min) | (L/min) | CFU/Puff |
| −2 | 1 | 18.1 | 11300 | 2 | 12.5 | 204530 |
| −3 | 3 | 17.92 | 833 | 2 | 12.5 | 14933 |
| −4* | 5 | 18.11 | 126 | 2 | 12.5 | 2294 |
| −4 | 6 | 18.13 | 190 | 2 | 12.5 | 3445 |
| −5 | 7 | 18.43 | 20 | 2 | 12.5 | 369 |

*Portion of spray impacted side of flow tube

The value under the heading “Puffer” indicates the exponent value of the dilution used (i.e. Puffer −2 refers to a 10⁻² dilution, etc.). These data demonstrate that the Bg particles present in the sample were stably and efficiently disseminated using the teachings of the present invention.

It is contemplated that many additions and modifications may be made to the present invention without departing from the spirit and scope of the present invention. Such additions and modifications will be readily apparent to those skilled in the art upon reading this disclosure. The disclosure above is provided to illustrate certain embodiments of the present invention and should not be construed as limiting the scope of the present invention, which is limited only by the claims below.

1. A propellant formulation for dispersing a plurality of insoluble particles, said particles having biological activity, for non-pharmaceutical uses, said propellant formulation having a specific gravity substantially the same as that of the plurality of insoluble particles, said propellant formulation further being chemically compatible with said plurality of insoluble particles.

2. The propellant formulation of claim 1 wherein said insoluble particles are bacterial spores.

3. The propellant formulation of claim 2 wherein said bacterial spores are Bacillus globigii spores.

4. The propellant formulation of claim 1 wherein said insoluble particles are biological analogues.

5. The propellant formulation of claim 1 wherein said formulation further has a vapor pressure sufficient to prevent agglomeration of said plurality of insoluble particles upon dispersion of insoluble particles.

6. The propellant formulation of claim 1 wherein said propellant formulation comprises a first propellant having a specific gravity greater than that of said plurality of insoluble particles, and a second propellant having a specific gravity less than that of said plurality of insoluble particles.

7. The propellant formulation of claim 1 wherein said plurality of insoluble particles to be dispersed by said propellant formulation is provided in a carrier.

8. The propellant formulation of claim 7 wherein said propellant formulation further includes a co-solvent.

9. A propellant formulation for dispersing a plurality of biological analogues, said propellant formulation having a specific gravity substantially the same as that of the plurality of biological analogues, said propellant formulation further being chemically compatible with said plurality of biological analogues.

10. The propellant formulation of claim 9 further having a vapor pressure sufficient to prevent agglomeration of said plurality of biological analogues upon dispersion of said biological analogues.

11. The propellant formulation of claim 9 wherein said propellant formulation comprises a first propellant having a specific gravity greater than that of said plurality of biological analogues, and a second propellant having a specific gravity less than that of said plurality of biological analogues.

12. A propellant formulation for dispensing at least one biological analogue, said formulation comprising:
a) a first propellant having a specific gravity greater than that of said at least one biological analogue; and
b) a second propellant having a specific gravity less than that of said at least one biological analogue;
wherein the specific gravity of said propellant formulation is substantially the same as that of the at least one biological analogue, and further wherein said propellant formulation is chemically compatible with said at least one biological analogue.

13. The propellant formulation of claim 12 wherein said at least one biological analogue to be dispersed by said propellant formulation is provided in a carrier.

14. The propellant formulation of claim 13 wherein said carrier is propylene glycol and wherein said propellant further includes a co-solvent.

15. The propellant formulation of claim 14 wherein said co-solvent is ethanol.

16. The propellant formulation of claim 12 wherein said first propellant is tetrafluroethane.

17. The propellant formulation of claim 16 wherein said second propellant is isobutane.

18. The propellant formulation of claim 17 wherein said at least one biological analogue is provided in a carrier.

19. The propellant formulation of claim 18 wherein said carrier is propylene glycol and wherein said propellant further includes a co-solvent.

20. The propellant formulation of claim 19 wherein said co-solvent is selected from the group consisting of methanol, ethanol, propanol, and butanol.

21. The propellant formulation of claim 19 wherein said co-solvent is selected from the group consisting of one-carbon alcohols, two-carbon alcohols, three-carbon alcohols, and four-carbon alcohols.

22. The propellant formulation of claim 12 wherein said propellant formulation is chemically compatible with a metered-dose inhaler.

23. The propellant formulation of claim 12 wherein said first propellant is selected from the group consisting of tetrafluoroethane, isobutane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and mixtures thereof.

24. The propellant formulation of claim 12 wherein said second propellant is selected from the group consisting of tetrafluoroethane, isobutane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and mixtures thereof.

25. A propellant formulation for dispersing at least one biological analogue, said formulation having a specific gravity of 1.06 at 70 degrees Fahrenheit and a vapor pressure of 62 psia at 70 degrees Fahrenheit, said formulation being chemically compatible with said biological analogue.

26. A propellant formulation for dispersing at least one biological analogue, said formulation comprising:
a) about 85.4% by weight tetrafluoroethane;
b) about 10.5% by weight isobutane;
c) about 3.46% by weight ethanol; and
d) about 0.63% by weight propylene glycol.

27. A method of dispersing a plurality of biological analogues, said method comprising:
a) providing said plurality of biological analogues in a carrier;
b) placing said biological analogues contained within said carrier into a metered-dose inhaler;
c) providing a propellant formulation having a specific gravity substantially the same as that of said plurality of biological analogues;
d) placing said propellant formulation within said metered-dose inhaler such that said biological analogues are dispersed into said propellant formulation; and
e) actuating said metered-dose inhaler to disperse said plurality of biological analogues.

28. The method of claim 27 wherein said propellant formulation has a vapor pressure sufficient to prevent agglomeration of said plurality of biological analogues upon dispersion of said plurality of biological analogues.

29. The method of claim 27 wherein said carrier is propylene glycol.

30. The method of claim 27 wherein said propellant formulation includes a co-solvent.

31. The method of claim 30 wherein said co-solvent is ethanol.

32. The method of claim 27 wherein said propellant formulation comprises a first propellant having a specific gravity greater than that of said plurality of biological analogues, and a second propellant having a specific gravity less than that of said plurality of biological analogues.

33. The method of claim 32 wherein said first propellant is selected from the group consisting of tetrafluoroethane, isobutane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and mixtures thereof.

34. The method of claim 32 wherein said second propellant is selected from the group consisting of tetrafluoroethane, isobutane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and mixtures thereof.

35. A propellant formulation for dispersing a biological analogue contained within a carrier, said formulation comprising, by weight:
a) 85.94% tetrafluoroethane;
b) 10.58% isobutane; and
c) 3.45% ethanol.

36. The formulation of claim 35 wherein said carrier is propylene glycol.

37. A propellant formulation for dispersing a plurality of biological analogues contained within a carrier, said formulation comprising:
a) a first propellant having a specific gravity greater than that of said plurality of biological analogues;
b) a second propellant having a specific gravity less than that of said plurality of biological analogues; and
c) a co-solvent for co-solving said carrier within said propellant formulation.

38. The propellant formulation of claim 37 further having a vapor pressure sufficient to prevent agglomeration of said plurality of biological analogues upon dispersion of said biological analogues.

39. The propellant formulation of claim 37 wherein said carrier is propylene glycol.

40. The propellant formulation of claim 37 wherein said co-solvent is ethanol.

41. The propellant formulation of claim 37 wherein said formulation has substantially the same specific gravity as said plurality of biological analogues.

42. The propellant formulation of claim 37 wherein said first propellant is selected from the group consisting of tetrafluoroethane, isobutane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and mixtures thereof.

43. The propellant formulation of claim 37 wherein said second propellant is selected from the group consisting of tetrafluoroethane, isobutane, heptafluoropropane, trichlorofluoromethane, dichlorofluoromethane, dichlorotetrafluoroethane, CF₃CF₃, CF₃CF₂CF₃, CHF₂CHF₂, CF₃CH₂F, CH₂F₂CH₃, CF₃CHFCF₃, and mixtures thereof.

44. The propellant formulation of claim 37 wherein said formulation is chemically compatible with a metered-dose inhaler.

45. The propellant formulation of claim 37 wherein said formulation has a specific gravity of 1.06 at 70 degrees Fahrenheit and a vapor pressure of 62 psia at 70 degrees Fahrenheit.
